(19) United States
(12) Patent Application Publication
(10) Pub. No.: US 2025/0341484 A1
(43) Pub. Date: Nov. 6, 2025

(54) MEASURING THERMAL EXPANSION AND THERMAL CONTRACTION COEFFICIENTS OF VISCOELASTIC MATERIALS

(71) Applicants: Jolina Karam, Phoenix, AZ (US); Kamil Elias Kaloush, Scottsdale, AZ (US)

(72) Inventors: Jolina Karam, Phoenix, AZ (US); Kamil Elias Kaloush, Scottsdale, AZ (US)

(21) Appl. No.: 19/197,389

(22) Filed: May 2, 2025

Related U.S. Application Data
(60) Provisional application No. 63/641,760, filed on May 2, 2024.

(51) Int. Cl. G01N 25/16; G01N 1/36; G01N 33/42

(52) U.S. Cl. CPC G01N 25/16; G01N 1/36; G01N 33/42; G01N 2001/366

(57) ABSTRACT

Determining a coefficient of thermal expansion or contraction of a sample includes coupling a strain gauge to a first location of the sample, coupling a thermocouple to a second location of the sample, placing the sample in a temperature-controlled chamber, increasing a temperature of the temperature-controlled chamber from a first temperature to a second temperature in a first cycle, increasing a temperature of the temperature-controlled chamber from a third temperature to a fourth temperature in a second cycle, assessing an average strain of the sample and an average temperature of the sample during the first cycle and the second cycle, and dividing a difference in the average strain of the sample between the first cycle and the second cycle by a difference in the average temperature of the sample between the first cycle and the second cycle to yield the coefficient of thermal expansion or contraction.

### CROSS-REFERENCE TO RELATED APPLICATION

This application claims the benefit of U.S. Patent Application No. 63/641,760 filed on May 2, 2024, which is incorporated by reference herein in its entirety.

### TECHNICAL FIELD

This invention relates to measuring thermal expansion/contraction coefficients for viscoelastic materials, with a particular emphasis on polymer-based and asphaltic materials used in various engineering applications.

### BACKGROUND

Thermal expansion describes the tendency of materials to change shape, area, volume, or density in response to temperature changes, as heat causes molecules to move more vigorously and occupy more space. The coefficient of thermal expansion (CTE) is a property of materials which refers to the rate at which a material expands with an increase in temperature. CTE is determined at a constant pressure without a phase change within the material. CTE depends on the bond strength between the atoms that make up the material. Covalent materials, such as diamond and crystals, have strong bonds between the atoms resulting in low CTEs. Thermal contraction is the reduction in a material's dimensions and volume that occurs when a material is subjected to a decrease in temperature.

Viscoelasticity refers to the behavior of materials that demonstrate both viscous and elastic qualities during deformation. Viscosity measures a fluid's resistance to deformation at a specific rate, while elasticity describes the capacity of a material to withstand distortions and return to its original size and shape once the distorting force is removed.

To determine the CTE and the coefficient of thermal contraction (CTC), displacement and temperature are measured on a sample undergoing a thermal cycle. Current methodologies primarily rely on dilatometry, a technique that requires materials to be in a fully fluid state, making it unsuitable for solid and semi-solid viscoelastic materials such as asphalt binders, crack sealants, and polymer-modified coatings.

### SUMMARY

This disclosure describes a method for measuring the coefficient of thermal expansion (CTE) and the coefficient of thermal contraction (CTC) of viscoelastic materials. The method includes preparing a sample and attaching a strain gauge and a thermocouple on a surface of the sample. The sample is placed into a temperature-controlled chamber and is measured at various temperatures. The change in strain of the sample is divided by the change in temperature of the sample to determine the CTE and CTC values of the sample.

In a first general aspect, determining a coefficient of thermal expansion or contraction of a sample includes coupling a strain gauge to a first location of the sample, coupling a thermocouple to a second location of the sample, placing the sample in a temperature-controlled chamber, increasing a temperature of the temperature-controlled chamber from a first temperature to a second temperature in a first cycle, increasing a temperature of the temperature-controlled chamber from a third temperature to a fourth temperature in a second cycle, assessing an average strain of the sample and an average temperature of the sample during the first cycle and the second cycle, and dividing a difference in the average strain of the sample between the first cycle and the second cycle by a difference in the average temperature of the sample between the first cycle and the second cycle to yield the coefficient of thermal expansion or contraction.

Implementations of the first general aspect can include one or more of the following features.

In some cases, the first general aspect further includes preparing the sample before coupling the strain gauge to the first location of the sample. Preparing the sample can include pouring a material into a mold. In some implementations, preparing the sample further includes curing the material. The mold can include a bending beam rheometer mold. A suitable example of the material includes a viscoelastic material. In certain cases, the viscoelastic material includes a polymer, a bituminous material, an asphalt-based material, or any combination thereof. The first general aspect can further include cleaning a surface of the sample with a catalyst (e.g., bonding adhesive or surface treatment/primer) before coupling the strain gauge to the first location.

In some cases, coupling the strain gauge to the first location includes adhering the strain gauge to the sample with an epoxy glue, a cyanoacrylate adhesive, or any combination thereof. In some implementations, coupling the thermocouple to the second location includes adhering the thermocouple to the sample with an epoxy glue, a cyanoacrylate adhesive, or any combination thereof. The thermocouple can be in direct contact with the sample. In certain cases, placing the sample in the temperature-controlled chamber includes placing a fluoropolymer sheet underneath the sample.

Increasing the temperature of the temperature-controlled chamber can include increasing the temperature at predetermined intervals for a length of time. In some implementations, the predetermined interval is in a range of 30 minutes to 2 hours. The increasing the temperature can include heating in a temperature range of 10° C. to 125° C. In certain implementations, increasing the temperature includes heating in a temperature range of 10° C. to 50° C. The first general aspect can further include, in the first cycle, allowing the sample to equilibrate at the second temperature. The first general aspect can further include, in the second cycle, allowing the sample to equilibrate at the fourth temperature. In some cases, the first general aspect further includes allowing the sample to cool between the first cycle and the second cycle. The second temperature can be greater than the third temperature.

The details of one or more embodiments of the subject matter of this disclosure are set forth in the accompanying drawings and the description. Other features, aspects, and advantages of the subject matter will become apparent from the description, the drawings, and the claims.

### BRIEF DESCRIPTION OF DRAWINGS

FIG. 1 is a flow chart showing operations in an example process for determining the CTE and the CTC for a material.

FIG. 2 shows measured strain values with increasing temperatures.

FIG. 3 shows the corresponding temperatures at which the strain values of FIG. 2 were measured.

FIG. 4 shows a CTE calibration curve.

### DETAILED DESCRIPTION

This disclosure describes a method for measuring the coefficient of thermal expansion (CTE) and the coefficient of thermal contraction (CTC) for viscoelastic materials. Examples of viscoelastic materials include polymers and bituminous and asphalt-based materials. These materials are typically used in roadway maintenance, roofing shingles, and polymeric surface coatings. Examples of asphalt-based materials can include asphalt crack sealants, asphalt binders, and surface treatments. Viscoelastic materials are temperature susceptible, where they soften at increased temperatures and harden at lower temperatures. Measuring CTE and CTC can compare how viscoelastic materials react under different temperatures and their behaviors can be used to improve compatibility between the binder, sealant, and the asphalt itself, preventing stress cracks and premature failure in pavements.

Reference test standards measure the CTE and CTC with a dilatometer, where the sample is typically in a fluid state. For viscoelastic materials, dilatometers may not capture the full response of the material until heated to an elevated temperature. This method installs strain gauges on a surface that can operate under various temperatures depending on the consistency of the sample.

Strain gauges are used to measure the movement of a material at different temperature cycles when placed in a controlled conditioning chamber. The strain differences can be recorded by a data acquisition system (e.g., Lab VIEW). In some cases, a Teflon sheet is placed underneath the sample to minimize the friction between the sample and the bottom of the chamber. The difference in strain due at least in part to the temperature cycling, divided by the difference in measured temperatures is used to calculate the coefficient of linear expansion and contraction. The geometry of a material is also evaluated to assess the expansion and contraction mechanisms.

A calibration round is conducted using different materials of well-known coefficients to calibrate the difference in measurement readings. The calibration test provides an insight into the material's ability to handle temperature changes by preserving its stability and integrity. If the material is opposing the movement of the pavement or the crack, the material's integrity can decrease, leading to premature failures.

The method includes two strain gauges where one is coupled to a sample and the other is coupled to a material of well-known thermal coefficient. When a resistance strain gauge is installed on a stress-free sample and its temperature is changed, the output of the gauge changes. The output of the resistance strain gauge changing in response to a changing resistance on the stress-free sample is referred to as temperature induced apparent strain or thermal output. The resistivity of the grid alloy of the gauge can change with the change in temperature. With this change, the grid is mechanically strained by an amount equal to the difference in expansion coefficients. Since the gauge grid is made from a strain sensitive alloy, it produces a resistance change proportional to the thermally induced strain.

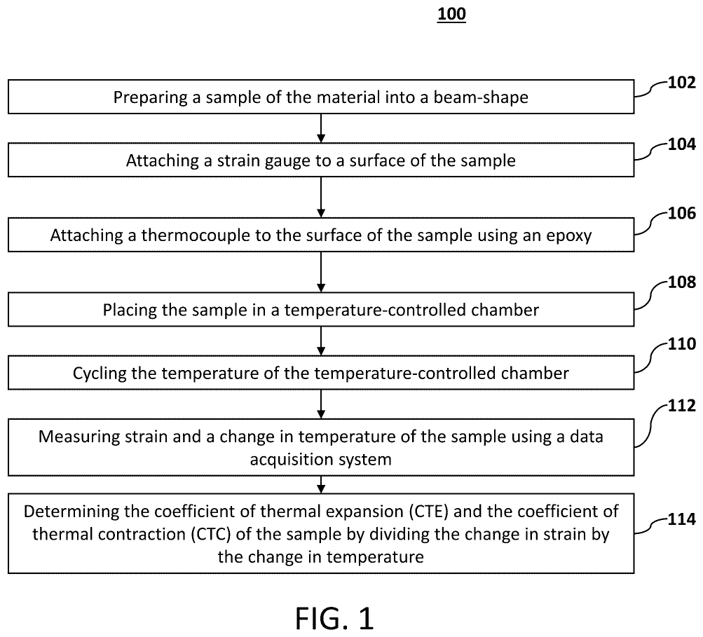

FIG. 1 is a flow chart showing operations in process 100 for determining the CTE and the CTC for a material. In 102, a material is prepared into a beam-shaped sample (e.g., 127 mm long, 6.35 mm wide, and 12.7 mm deep). A suitable example for the material includes viscoelastic materials. Examples of viscoelastic materials include polymers, bituminous materials, and asphalt-based materials (e.g., asphalt crack sealants, asphalt binders, and surface treatments). In some cases, the samples are prepared by making the materials liquid enough to be poured into the beam-shaped molds. The mold is typically greased prior to pouring the materials to allow for ease of removal. The samples are typically left to cure overnight at room temperature, allowing the settlement of the material. The samples are typically trimmed and flipped over to reveal a surface that was not in contact with the mold.

In 104, a strain gauge is attached to a surface of the sample. Before adhering the strain gauge on the sample, the gauge is typically checked to ensure it is working properly. A multimeter is typically used to measure the resistance differences between the three cables (red, white, and black). The measured values are typically 0 ohms for black and white cables, 120 ohms for red and white cables, and 120 ohms for red and black cables. If the measurements are different, the gauge is typically considered faulty and is discarded. The surface of the sample is typically cleaned with a bonding adhesive that serves as a surface treatment or binder (e.g., M-BOND 200 Catalyst C) to allow proper bonding between sample and gauge. The strain gauge is typically fixed on a piece of tape to attach it on a desired location on the sample. The side of the strain gauge that does not touch the sample is typically fixed on the tape. A drop of cyanoacrylate adhesive is typically spread on the opposite side of the gauge and is then typically pressed on the surface of the sample for about one minute to allow the cyanoacrylate adhesive to cure. The samples typically remain in the mold until the samples are ready to be measured. After approximately one minute, the tape is typically gently peeled out of the surface of the sample at an angle of 45°, leaving the gauge in position.

In 106, a thermocouple is attached to the surface of the sample using an epoxy. In some cases, the epoxy is cured for about 6 hours. The tip of the thermocouple typically touches the surface of the sample. If the tip of the thermocouple touches the epoxy, the temperature measurements recorded are typically based on the properties of the epoxy and not the sample to be assessed.

In 108, the sample is placed in a temperature-controlled chamber. In some cases, the sample is placed inside the chamber with a Teflon sheet underneath. This sheet can minimize the friction underneath the sample. In 110, the temperature of the temperature-controlled chamber is cycled. The temperature is typically set to increase or decrease at specific intervals. Once the selected temperature is reached, a 45-minute duration is typically allocated to allow the sample to reach equilibrium at that temperature, known as a steady-state. In 112, strain and a change in temperature of the sample are measured using a data acquisition system (e.g., Lab VIEW software). In 114, the CTE and the CTC of the sample are determined by dividing the change in strain by the change in temperature. From each interval for both temperature and strain, average values are typically calculated. The change in strains Δε is typically calculated by subtracting the average strain values obtained from the previous thermal cycle and the average strain values obtained from the subsequent thermal cycle. A calibration round is typically conducted using different materials of well-known coefficients to adjust the difference in measurement readings.

### EXAMPLES

The equipment used to measure the expansion and contraction of the viscoelastic materials include strain gauges (C4A-06-235SL-120-39P, Micro-Measurements), thermocouple type K, epoxy (KwikWeld J-B Weld 8276b), cyanoacrylate adhesive gel, Teflon sheets, Lab VIEW Software with Data Acquisition System by National Instruments, Bending Beam Rheometer (BBR) molds for sample preparation, and a temperature controlled chamber.

The sample size was chosen based on the size used during a BBR test (AASHTO TP 87, 2015) (127 mm long, 6.35 mm wide and 12.7 mm deep). In some cases, the thickness of the material affected the reading while the length had negligible impact.

The samples were prepared by making the materials liquid enough to be poured in the beam-shaped molds. The mold was greased prior to pouring the materials to allow for removal of the molds at the time of measuring. The samples were left to cure overnight at room temperature, allowing the settlement of the material.

Before adhering the strain gauge on the sample, the gauge was checked to ensure it was working properly. By using a multimeter, the resistance difference between the three cables (red, white, and black) were measured as follows: black and white cables (0 ohms), red and white cables (120 ohms), and red and black cables (120 ohms). If the measurements were different, the gauge was considered faulty and was discarded.

The samples were trimmed and the strain gauges were placed on the smooth side of the beam by flipping the sample on the side that was in contact with the mold. The surface of the sample was cleaned with a catalyst (e.g., M-BOND 200 Catalyst C) to allow proper bonding between sample and gauge. The strain gauge was fixed on a piece of tape to attach it on a desired location on the sample. The side of the strain gauge that did not touch the sample was fixed on the tape. A drop of cyanoacrylate adhesive was spread on the opposite side of the gauge and was then pressed on the surface of sample for about one minute to allow the cyanoacrylate adhesive to cure. The samples remained in the mold until the samples were ready to be measured. After one minute, the tape was gently peeled out of the surface of the sample at an angle of 45°, leaving the gauge in position.

The thermocouples were subsequently placed at the surface of the sample to measure the temperature of the sample at every cycle. The thermocouples were fixed to the surface of the sample using epoxy glue with a curing time of 6 hours. The tip of the thermocouple touched the surface of the sample. If the tip of the thermocouple touched the epoxy, the temperature measurements recorded were based on the properties of the epoxy and not the sample to be measured. The thermocouple was typically placed close to the strain gauge to capture the temperature at a location close to where the strain was being measured. After waiting for 6 hours, the sample was ready to be measured. The mold was removed from the sample and placed inside the chamber with a Teflon sheet underneath. This sheet minimized the friction underneath the sample and allowed the sample to be less restricted.

The sample was subjected to thermal cycling in a temperature-controlled chamber. Temperature was set to increase or decrease at specific intervals. Once the selected temperature was reached, a 45-minute duration was allocated to allow the sample to reach equilibrium at that temperature, known as steady-state. The strain gauge measured the strains at different temperatures associated with the resulting expansion or contraction of the material undergoing thermal cycling. Furthermore, the use of strain gauges could avoid the limitations associated with shape and geometry of the measured material. As expansion and contraction occurs, the material's shape could be deformed at elevated temperatures. In the case of strain gauges, the difference in strains due at least in part to thermal cycling was measured at the strain gauge boundaries.

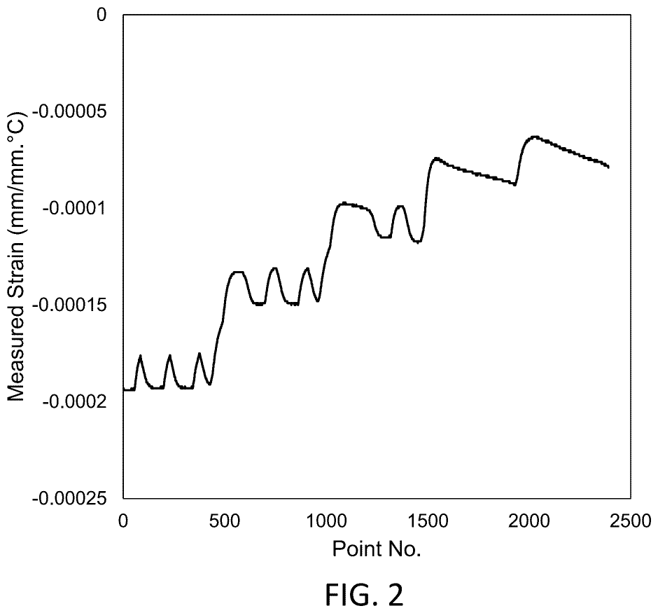

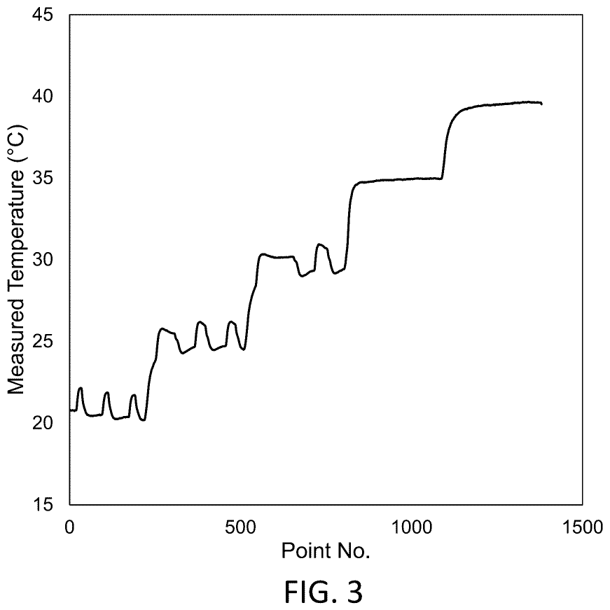

An example of measured and recorded strain values of an asphalt crack sealant material are shown in FIG. 2, where the measured strain increases with increasing temperatures. The corresponding temperatures at which those strains were measured are shown in FIG. 3.

From each interval for both temperature and strains, average values were considered for further calculations. The change in strains Δε was calculated by subtracting the average strain values obtained from the previous thermal cycle and the average strain values obtained from the subsequent thermal cycle.

Table 1 shows the average temperature, average strain, and calculated CTE for each cycle of the five total cycles. The CTE was calculated according to Equation 1.

$$α = \frac{Δ ε}{Δ T} \qquad ( Equation 1 )$$

In Equation 1, α is the CTE or the CTC (mm/mm ° C.), ΔT is the change in temperature (° C.), either increasing for expansion or decreasing for contraction, and Δε is the change in strain during the same period caused by either the expansion or contraction of the material.

TABLE 1
Average temperature, average strain, and
CTE of sample at each thermal cycle

| | Average | | |
|---|---|---|---|
| Cycle | Temperature | Average Strain | Calculated |
| Number | (° C.) | (mm/mm · ° C.) | CTE |
| 1 | 20.803 | −0.00018787 | 1.06561E−05 |
| 2 | 25.112 | −0.000141951 | 7.39888E−06 |
| 3 | 29.895 | −0.000106566 | 4.90914E−06 |
| 4 | 34.898 | −8.2003E−05 | 2.70757E−06 |
| 5 | 39.125 | −7.05586E−05 | |

The average of the calculated CTE values was considered to be the final value, representative of the measured sample. In this example, the average CTE value was 6.42×10⁻⁶. A calibration chart, obtained after measuring different materials of known CTE, was used to reflect a final value of 1.85×10⁻⁵.

To determine if geometry of the sample affected the measurements, beams with different lengths were measured using the same conditions at the same time in the conditioning chamber. The initial length denoted as “Big” reflected the original size of the sample of 127 mm. The other length denoted as “Small” referred to half of the initial length. The results in Table 2 showed that the two different lengths of the samples resulted in similar measured values. Therefore, the original length of the sample was adopted for the remaining measurements. The width of the sample was chosen to be wide enough to provide a surface for the placement of the gauge. Since the linear expansion in the direction of the gauge was of interest, the width of the sample was minimized to reduce errors in measurements. Additionally, the width of the sample was minimized to deter expansion, contraction, or both in other directions of the material. The materials of interest were anisotropic due at least in part to the modifiers implemented and the nature of the material itself.

TABLE 2
Results of the CTE Geometry Analysis

| Materials with | Average | | |
|---|---|---|---|
| Different | Measurements | Reference Values | |
| Lengths | (mm/mm · ° C.) | (mm/mm · ° C.) | COV |
| Big Aluminum | 1.08E−05 | 2.36E−05 | 74% |
| Small Aluminum | 9.38E−06 | 2.36E−05 | 86% |
| Big Brass | 7.38E−06 | 1.87E−05 | 87% |
| Small Brass | 6.72E−06 | 1.87E−05 | 94% |

A 12 mm thickness was considered to be representative of the material's thickness that includes the possibility of having a number of modifiers of different sizes (e.g., fibers, rubber). In this example, the samples' CTE was measured for temperature cycles ranging from 20° C. to 40° C., where negligible changes to the geometry due at least in part to softening were observed. For this reason, the change in geometry was not considered.

Different materials with well-known CTE were measured using the suggested setup. The measurements obtained were then compared to reference values. The materials were obtained in the same geometry (127 mm long, 6.35 mm wide and 12.7 mm deep):

TABLE 3
Selected samples for CTE calibration

| | | Coefficient of |
|---|---|---|
| Sample | | Linear Expansion |
| Measured | Characteristics | (mm/mm · ° C.) |
| Aluminum | Multipurpose, 6061 | 2.36E−05 |
| Brass | Ultra-machinable, 360 | 1.87E−05 |
| Acrylic | Clear Scratch, UV-Resistant Cast | 7.40E−05 |
| Steel | Tight-Tolerance Low-Carbon, A36 | 1.20E−05 |
| Stainless | Multipurpose, 304 | 1.72E−05 |
| Steel | | |

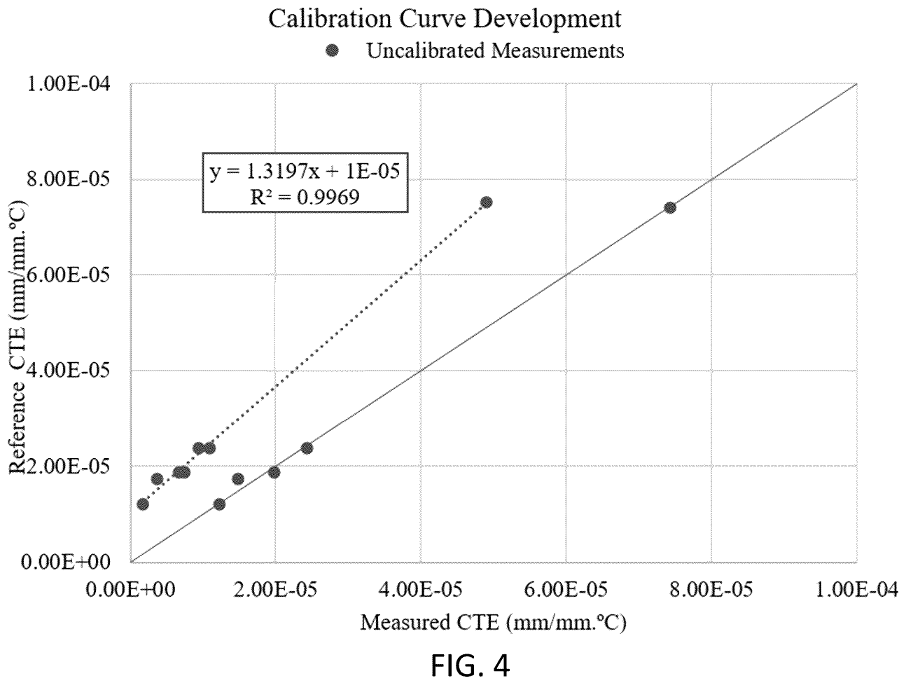

The linear expansion coefficients for the reference materials were typically obtained for expansion between 20° C. and 100° C. For this reason, this method was used to measure both expansion and contraction using a similar temperature range from 20° C. to 100° C. A calibration curve shown in FIG. 4 was developed by comparing the measured values to the reference values for all the materials listed in Table 3. FIG. 4 shows the uncalibrated CTE values for the reference materials, where a dotted linear trendline was fitted to the data. An R² value of 0.9969 suggested that a linear trendline was appropriate with respect to the obtained data. Then, the uncalibrated values were adjusted and shown in the same figure. The results of the calibration process are shown in Table 4. The coefficient of variation (COV) between the calibrated and reference values for the selected materials was acceptable with a maximum of 15% difference for stainless steel.

TABLE 4
CTE calibration results

| | Uncalibrated | Calibrated | Reference | |
|---|---|---|---|---|
| | Measurements | Measurements | Values | |
| | (mm/mm · | (mm/mm · | (mm/mm · | |
| Materials | ° C.) | ° C.) | ° C.) | COV |
| Aluminum | 1.08E−05 | 2.43E−05 | 2.36E−05 | −3% |
| Brass | 7.38E−06 | 1.97E−05 | 1.87E−05 | −5% |
| Acrylic | 4.87E−05 | 7.42E−05 | 7.40E−05 | 0% |
| Steel | 1.71E−06 | 1.23E−05 | 1.20E−05 | −2% |
| Stainless | 3.61E−06 | 1.48E−05 | 1.72E−05 | 15% |
| Steel | | | | |

Although this disclosure contains many specific embodiment details, these should not be construed as limitations on the scope of the subject matter or on the scope of what may be claimed, but rather as descriptions of features that may be specific to particular embodiments. Certain features that are described in this disclosure in the context of separate embodiments can also be implemented, in combination, in a single embodiment. Conversely, various features that are described in the context of a single embodiment can also be implemented in multiple embodiments, separately, or in any suitable sub-combination. Moreover, although previously described features may be described as acting in certain combinations and even initially claimed as such, one or more features from a claimed combination can, in some cases, be excised from the combination, and the claimed combination may be directed to a sub-combination or variation of a sub-combination.

Particular embodiments of the subject matter have been described. Other embodiments, alterations, and permutations of the described embodiments are within the scope of the following claims as will be apparent to those skilled in the art. While operations are depicted in the drawings or claims in a particular order, this should not be understood as requiring that such operations be performed in the particular order shown or in sequential order, or that all illustrated operations be performed (some operations may be considered optional), to achieve desirable results.

Accordingly, the previously described example embodiments do not define or constrain this disclosure. Other changes, substitutions, and alterations are also possible without departing from the spirit and scope of this disclosure.

What is claimed is:

1. A method for determining a coefficient of thermal expansion or contraction of a sample, the method comprising:
coupling a strain gauge to a first location of the sample;
coupling a thermocouple to a second location of the sample;
placing the sample in a temperature-controlled chamber;
in a first cycle, increasing a temperature of the temperature-controlled chamber from a first temperature to a second temperature;
in a second cycle, increasing a temperature of the temperature-controlled chamber from a third temperature to a fourth temperature;
assessing an average strain of the sample and an average temperature of the sample during the first cycle and the second cycle; and
dividing a difference in the average strain of the sample between the first cycle and the second cycle by a difference in the average temperature of the sample between the first cycle and the second cycle to yield the coefficient of thermal expansion or contraction.

2. The method of claim 1, further comprising preparing the sample before coupling the strain gauge to the first location of the sample.

3. The method of claim 2, wherein preparing the sample comprises pouring a material into a mold.

4. The method of claim 3, wherein preparing the sample further comprises curing the material.

5. The method of claim 3, wherein the mold comprises a bending beam rheometer mold.

6. The method of claim 1, wherein the material is a viscoelastic material.

7. The method of claim 6, wherein the viscoelastic material comprises a polymer, a bituminous material, an asphalt-based material, or any combination thereof.

8. The method of claim 1, further comprising cleaning a surface of the sample with a catalyst before coupling the strain gauge to the first location.

9. The method of claim 1, wherein coupling the strain gauge to the first location comprises adhering the strain gauge to the sample with an epoxy glue, a cyanoacrylate adhesive, or any combination thereof.

10. The method of claim 1, wherein coupling the thermocouple to the second location comprises adhering the thermocouple to the sample with an epoxy glue, a cyanoacrylate adhesive, or any combination thereof.

11. The method of claim 1, wherein the thermocouple is in direct contact with the sample.

12. The method of claim 1, wherein the placing the sample in the temperature-controlled chamber comprises placing a fluoropolymer sheet underneath the sample.

13. The method of claim 1, wherein increasing the temperature of the temperature-controlled chamber comprises increasing the temperature at predetermined intervals for a length of time.

14. The method of claim 13, wherein the predetermined interval is in a range of 30 minutes to 2 hours.

15. The method of claim 1, wherein increasing the temperature comprises heating in a temperature range of 10° C. to 125° C.

16. The method of claim 15, wherein increasing the temperature comprises heating in a temperature range of 10° C. to 50° C.

17. The method of claim 1, further comprising, in the first cycle, allowing the sample to equilibrate at the second temperature.

18. The method of claim 1, further comprising, in the second cycle, allowing the sample to equilibrate at the fourth temperature.

19. The method of claim 1, further comprising allowing the sample to cool between the first cycle and the second cycle.

20. The method of claim 1, wherein the second temperature is greater than the third temperature.
